(19) United States
(12) Patent Application Publication
(10) Pub. No.: US 2005/0287199 A1
(43) Pub. Date: Dec. 29, 2005

(54) Therapeutic micro nutrient composition for lipolysis and drug delivery

(76) Inventors: Teresa Ann Denney, Honolulu, HI (US); James Robert DeLuze, Honolulu, HI (US)

Correspondence Address: Teresa Ann Denney;Suite 106, 4218 Waialae Avenue, Honolulu, HI, 96816, US

(21) Appl. No.: 10/881,170

(22) Filed: Jun. 28, 2004

(51) Int. Cl. A61K 31/685; A61K 31/355; A61K 9/127

(52) U.S. Cl. 424/450; 514/78; 514/458

(57) ABSTRACT

An improved formulation and method for the removal of subcutaneous fat deposits in a human in need of such treatment. A lecithin based biphasic injection dosage formulation is disclosed which is applicable to subcutaneous, intramuscular, and intravenous administration. Additionally, a program based approach to the treatment of subcutaneous fat deposits which includes injections of this formulation, application of compression garments, diet modification, and exercise is described. The formulation is characterized in that it comprises an adjustable buffer, an antioxidant, and a stabilizer. It is further characterized in that it includes liposomes, and that the components of these liposomes are therapeutic in the treatment of several human ailments. It is also efficacious in the treatment of striae albicantes, striae atrophicae, cellulite, and decreased skin turgor. In an alternate embodiment, the formulation is characterized in that it comprises a carrier of biologically active substances.

### BACKGROUND

1. Field of Invention

The present invention relates to a biphasic aqueous and lipophylic combination of phosphatidylcholine and sodium chloride. In particular, the invention relates to a biphasic injectable dosage form of phosphatidylcholine and other micro nutrients for the lipolysis of subcutaneous fat. The invention also is a new injectable dosage formulation for the delivery of drugs and other biologically active agents.

2. Description of Prior Art

Aqueous sodium chloride is commonly used as an injectable, parenteral, or enteral dosage formulation for medical treatment and as a delivery agent for other therapeutic agents.

Phosphatidylcholine is also used as an injectable, parenteral or enteral dosage formulation for treatment of various disorders.

Thereafter, inventors created a subcutaneous dosage formulation of erythropoietin utilizing phosphatidylcholine and other micro nutrients as an erythropoietin carrier. U.S. Pat. No. 6,645,522 to Naeff, Delmenico, Wetter, and Floether (2003) discloses a liposome based formulation of erythropoietin comprising: (a) erythropoietin; (b) a lipidic phase comprising: (i) lecithin; (ii) a charged lipid; and (iii) cholesterol; and (c) a phosphate buffer.

Thereafter, inventors created an oral dosage formulation of micro nutrients for treatment of severe trauma, burns, and critical illness. U.S. Pat. No. 6,391,332 patent to Somerville and Sherratt (2002) discloses a micro nutrient formulation containing water soluble and lipid soluble micro nutrients for oral and feeding tube administration.

### BACKGROUND OF THE INVENTION

Lecithin is a phospholipid which serves as a principal factor involved in the transport, regulation, and metabolism of fatty substances. It is a fatty food substance. It is a structural component of every cell in the body. It is an important component of cell membranes. It has been used as an effective treatment agent in the treatment of hypercholesteremia, hypertriglyceridemia, alcoholic hepatic steatosis, and xanthelasma. The phospholipid is administered orally or parenterally as either an intravenous (IV) or subcutaneous (SC) injection.

Presently, lecithin is injected SC for the reduction of subcutaneous fat deposits. This procedure was discovered by Brazilian dermatologist Patricia Rittes and is most commonly called Lipo-Disolve. In April 1999 the FDA approved a Baxter Healthcare Corporation product, Cernevit-12, which contains lecithin for injection. This product is a vitamin delivery system for parenteral nutrition.

While these formulations have been proven to be successful, further improvement for the treatment of subcutaneous fat deposits has been desired. The reduction of subcutaneous fat can leave the skin loose. The efficacy of the procedure could be significantly improved. Hence, there is a need for an improved phospholipid formulation for the treatment of subcutaneous fat deposits.

Accordingly, attempts have been made to provide an improved formulation of phospholipids which increases skin tone and proves to be more efficacious. At the same time the formulation should be stable and of the correct hydrogen ion concentration (pH).

A biphasic formulation comprises an an aqueous phase and a lipidic phase. This provides for the solubility of both lipid and aqueous soluble components. This formulation then can be a vehicle for the transport of both lipid and water soluble substances to the targeted treatment area.

The properties of such a biphasic formulation will provide conditions which favor the formation of liposomes. Liposomes are small vesicles comprising amphipathic lipids arranged in spherical bilayers. Liposomes may contain many concentric lipid bilayers separated by aqueous channels (multilamellar vesicles or MLVs), or alternatively, they may contain a single membrane bilayer (unilamellar vesicles), which may be small unilamellar vesicles (SUVs) or large unilamellar vesicles (LUVs). The lipid bilayer is composed of two lipid monolayers having a hydrophobic “tail” region and a hydrophilic “head” region. In the membrane bilayer, the hydrophobic “tails” of the lipid monolayers orient towards the center of the bilayer, whereas the hydrophilic “heads” orient towards the aqueous phase.

Liposomes may be used to encapsulate a variety of materials by trapping hydrophilic compounds in the aqueous interior or between bilayers, or by trapping hydrophobic compounds within the bilayer. As such, they are particularly useful to deliver biologically active materials by encapsulating compounds which exhibit poor aqueous solubility or which exhibit unacceptable toxicity at therapeutic dosages.

In addition, liposomes may be used to deliver biologically active materials which are at the same time components of the liposome itself. Such is the result of the formation of liposomes from phospholipids including lecithin and it's pharmaceutically acceptable derivatives.

The goal of this present invention therefore was to provide a parenteral formulation suitable for the treatment of subcutaneous fat deposits, provides for pH adjustment, has stability, and increases skin turgor.

### SUMMARY

A biphasic phospholipid based parental composition comprising:

(a) an effective amount of an active ingredient comprising a lipid, phospholipid, or phospholipids selected from the group consisting of lecithin, hydrogenated lecithin, phosphatidylcholine, hydrogenated phosphatidylcholine, and tocopherol, or their pharmaceutically acceptable derivatives. This active ingredient or active ingredients having the biological properties of causing the destruction of adipose cells and the lipolysis of the fatty material contained within the treated adipose tissue.

(b) a lipidic phase comprising:

(i) lecithin or hydrogenated lecithin;

(ii) phosphatidylcholine or hydrogenated phosphatidylcholine;

(iii) tocopherol;

(iv) optional lipid soluble components as described below in (d); and

(c) an aqueous phase comprising:

(i) water;

(ii) sodium chloride;

(iii) optional water soluble components as described below in (d); and

(d) biologically active substances as added to individualized treatment regimens as further selected from the group consisting of nutrients, micro nutrients, vitamins, and drugs.

In accordance with the invention, the selected components of the lipid phase are mixed with the selected components of the aqueous phase. The relative amounts of the compounds mixed is predetermined. The mixing of these lipid and water soluble components favors the formation of liposomes.

Liposomes may be used to encapsulate a variety of materials by trapping hydrophilic compounds in the aqueous interior or between bilayers, or by trapping hydrophobic compounds within the bilayer. As such, they are particularly useful to deliver biologically active materials by encapsulating compounds which exhibit poor aqueous solubility or which exhibit unacceptable toxicity at therapeutic dosages.

In addition, liposomes may be used to deliver biologically active materials which are at the same time components of the liposome itself. Such is the result of the formation of liposomes from phospholipids including lecithin and it's pharmaceutically acceptable derivatives.

In addition, a method of treatment for subcutaneous fat deposits has been developed which includes the following components:

(a) injection of the biphasic phospholipid based parental composition;

(b) application of a compression garment;

(c) a predetermined exercise program;

(d) a predetermined diet regimen.

In accordance with the present invention, it has been discovered that this biphasic phospholipid based parental composition described herein exhibits improved efficacy in the treatment of subcutaneous fat deposits. As further advantages, the presence of the sodium chloride acts as a mild sclerosing agent. This sclerosing action has the physiological function of increasing the adherence of subcutaneous layers of skin and fascia resulting in increased skin turgor. It has also been discovered that, quite unexpectedly, that this combination of fat reduction and mild sclerosing action is efficacious for the treatment of cellulite deposits.

### OBJECTS AND ADVANTAGES

Accordingly, besides the objects and advantages of this biphasic micro nutrient dosage formulation described in our above patent, several objects and advantages of the present invention are:

(a) to circumvent the bodies metabolic adjustments as part of the feast or famine cycle.

(b) to reduce the numbers of adipose cells in treated areas.

(c) to provide a biphasic injection nutrient delivery means.

(d) to provide an injectable, parenteral or enteral nutrient delivery means for aqueous soluble substances.

(e) to provide an injectable, parenteral or enteral nutrient delivery means for lipid soluble substances.

### DESCRIPTION OF INVENTION

A method for reduction of adipose tissue in humans comprising injection of a biphasic dosage formulation based on lecithin in an amount effective to stimulate lipase production in adipose tissue. Lipase and it's related compounds stimulate the lysis, destruction, and reduction of the amount of adipose cells. Lipase also stimulates the lipolysis of fatty material contained within adipose tissue.

This invention encompasses a systematic means of reduction of adipose tissue. It incorporates the biphasic dosage formulation based on lecithin, use of a compression garment, exercise management and counseling, and diet management and counseling. This multifaceted approach increases the successful removal of unwanted adipose tissue. This biphasic injection formulation is called Lipolyte.

Obesity is a serious medical problem resulting is significant morbidity and mortality. Many people spend hours exercising and try all kinds of diet regimens, but the obesity remains.

The underlying problem is that the body has an internal set point of how much fat composition is optimal. This set point is basically determined by the numbers of adipose cells present. This was determined by both genetic and environmental factors. The environmental factors include the behavior of a persons mother during pregnancy, behavior patterns during growth spurts during early childhood and at puberty, and continuing behavioral patterns into adulthood.

When a person looses weight through dieting and exercise, the body changes metabolic patterns in order to retain fat. This results in a continuous cycle of dieting and weight gain. This repetitive cycle also significantly contributes to morbidity and mortality as a result of high circulating fat levels in the blood.

This biphasic injection formulation circumvents this vicious cycle. Administration of Lipolyte has the biological property of reducing the number of adipose cells in the treated areas. In combination with exercise and appropriate diet modifications, the ongoing starvation verses feasting cycle is circumvented.

The principal active ingredient used in the present biphasic injection formulation is a lipid, phospholipid, or phospholipids selected from the group consisting of lecithin, hydrogenated lecithin, phosphatidylcholine, hydrogenated phosphatidylcholine, and tocopherol, or their pharmaceutically acceptable derivatives. This active ingredient or active ingredients have the biological properties of causing adipose cells and related tissue to release lipase and related substances. This released lipase, and related substances, have the biological properties of the lysis, destruction, and reduction of the amount of adipose cells in a given region. In addition lipase, and related substances, also has the biological properties resulting in lipolysis of fatty material contained within these adipose cells and adipose tissue.

Another active ingredient used in this biphasic injection formulation is aqueous sodium chloride. This active ingredient has the biological property of causing a mild tissue sclerosing effect. This sclerosing action has the physiological function of increasing the adherence of subcutaneous layers of skin and fascia resulting in increased skin turgor.

This biphasic phospholipid based parental composition of the present invention is useful as a parenteral formulation in treating subcutaneous fat deposits, decreased skin turgor, striae atrophicae, xanthelasma, striae albicantes, and subcutaneous cellulite deposits. It may also have application in the treatment of a variety of disease states, disorders, and states of hematologic irregularity such as atherosclerosis, diabetes, hypercholesteremia, hypertriglyceridemia, and alcoholic hepatic steatosis. It may also have application in the treatment of respiratory distress syndrome, necrotizing enterocolitis, central nervous system cholinergic imbalances, bipolar depression, alzheimer's disease, hepatitis B, hepatitis C, and other liver diseases.

A biphasic phospholipid based parental composition comprising:

(a) an effective amount of an active ingredient comprising a lipid, phospholipid, or phospholipids selected from the group consisting of lecithin, hydrogenated lecithin, phosphatidylcholine, hydrogenated phosphatidylcholine, and tocopherol, or their pharmaceutically acceptable derivatives. This active ingredient or active ingredients having the biological properties of causing the destruction of adipose cells and the lipolysis of the fatty material contained within the treated adipose tissue;

(b) a lipidic phase comprising:

(i) lecithin or hydrogenated lecithin;

(ii) phosphatidylcholine or hydrogenated phosphatidylcholine;

(iii) tocopherol;

(iv) optional lipid soluble components as described below in (d); and

(c) this lipid phase comprising 0.0 to 100 percent of the biphasic phospholipid based parental composition; and

(d) the individual lipids comprising the following fraction of the lipid phase:

(i) lecithin 0.0 to 100 percent;

(ii) hydrogenated lecithin 0.0 to 100 percent;

(ii) phosphatidylcholine 0.0 to 100 percent;

(iv) hydrogenated phosphatidylcholine 0.0 to 100 percent;

(v) tocopherol 0.0 to 100 percent;

(vi) optional water soluble components as described below in (g) 0.0 to 100 percent; and

(e) an aqueous phase comprising:

(i) water;

(ii) sodium chloride;

(iii) optional water soluble components as described below in (g); and

(f) the individual water soluble compounds comprising the following fraction of the lipid phase:

(i) water 0.0 to 100 percent;

(ii) sodium chloride 0.0 to 100 percent;

(iii) optional water soluble components as described below in (g) 0.0 to 100 percent; and

(g) biologically active substances as added to individualized treatment regimens as further selected from the group consisting of nutrients, micro nutrients, vitamins, and drugs.

Lecithin can be used as natural lecithin in purified sterile form or as the more stable hydrogenated lecithin, whereby the formulation is more stable. Lecithin is a phospholipid which serves as a principal factor involved in the transport, regulation, and metabolism of fatty substances. It is a fatty food substance. It is a structural component of every cell in the body. It is an important component of cell membranes. Presently, the phospholipid is administered orally or parenterally as either an intravenous (IV) or subcutaneous (SC) injection.

Presently, lecithin is injected SC for the reduction of subcutaneous fat deposits. This procedure was discovered by Brazilian dermatologist Patricia Rittes and is most commonly called Lipo-Disolve. In April 1999 the FDA approved a Baxter Healthcare Corporation product, Cernevit-12, which contains lecithin for injection. This product is a vitamin delivery system for parenteral nutrition.

A biphasic formulation comprises an an aqueous phase and a lipidic phase. This provides for the solubility of both lipid and aqueous soluble components. This formulation then can be a vehicle for the transport of both lipid and water soluble substances to the targeted treatment area.

The properties of such a biphasic formulation will provide conditions which favor the formation of liposomes. Liposomes are small vesicles comprising amphipathic lipids arranged in spherical bilayers. Liposomes may contain many concentric lipid bilayers separated by aqueous channels (multilamellar vesicles or MLVs), or alternatively, they may contain a single membrane bilayer (unilamellar vesicles), which may be small unilamellar vesicles (SUVs) or large unilamellar vesicles (LUVs). The lipid bilayer is composed of two lipid monolayers having a hydrophobic “tail” region and a hydrophilic “head” region. In the membrane bilayer, the hydrophobic “tails” of the lipid monolayers orient towards the center of the bilayer, whereas the hydrophilic “heads” orient towards the aqueous phase.

Liposomes may be used to encapsulate a variety of materials by trapping hydrophilic compounds in the aqueous interior or between bilayers, or by trapping hydrophobic compounds within the bilayer. As such, they are particularly useful to deliver biologically active materials by encapsulating compounds which exhibit poor aqueous solubility or which exhibit unacceptable toxicity at therapeutic dosages.

In addition, liposomes may be used to deliver biologically active materials which are at the same time components of the liposome itself. Such is the result of the formation of liposomes from phospholipids including lecithin and it's pharmaceutically acceptable derivatives.

This biphasic phospholipid based parental composition is appropriate to be delivered by subcutaneous, intravenous and intramuscular injection. A lecithin containing formulation was FDA approved for subcutaneous and intravenous injection in April of 1999 to Baxter Healthcare Corporation.

The compounds of the lipid phase constitute a stabilizer. Additionally, the components of both the lipid and aqueous phase together comprise a mild buffer. The set pH and capacity of this buffer can be adjusted to predetermined amounts by changing the relative amounts of the dosage formulation. This involves the adjustment of the relative amounts of the lipid and aqueous phases. It also involves the adjusting the relative amounts of the components of both the lipid and aqueous phases. This provides for the adjustment of the parameters of this buffering action to be tailored to most suit the given target area undergoing treatment by injection of the formulation.

Tocopherol, a compound component of the lipid phase is an anti oxidant. Local anesthetics lidocaine and bipivacaine are both stable and soluble in this formulation. They may be included to provide analgesia to the injected area.

This invention also comprises a program to facilitate the efficacy of the formulation. The dosage and given formulation injected in a given patient is to be guided by a physician skilled in the art on a case by case basis. Patients may receive multiple injections, appropriately spaced, to a given target area per day. This schedule of injections may be then repeated as per the physicians clinical judgment.

It has been found that the application of a compression garment to the treated area facilitates both the removal of subcutaneous fat and also the sclerosing action of the formulation. A moderate exercise program is also encouraged and guided by a physician skilled in the art on a case by case basis. Diet modification, education, and counseling is also guided by a physician skilled in the art on a case by case basis. To this end, a diet approximately equal in protein with complex carbohydrates and low fat is encouraged. The combination of injections, compression garment, diet, and moderate exercise has proven to be the most efficacious in the treatment of subcutaneous fat deposits.

As stated above, the lecithin based compositions marketed have some efficacy in the treatment of subcutaneous fat deposits. It has been found that the biphasic dosage formulation based on lecithin described above has enhanced efficacy in the treatment of subcutaneous fat deposits. Additionally, this formulation has shown efficacy in the treatment of other disorders as disclosed above.

The efficacy of treatment of subcutaneous fat deposits can be further enhanced by the addition of including such injections into a program including diet, exercise, and the wearing of a compression garment as guided by a physician skilled in the art on a case by case basis.

Operation of Invention and it's Alternative Embodiments

The present invention relates to a biphasic nutrient and micro nutrient composition which includes phospholipids and aqueous sodium chloride. The biphasic nature of this composition promotes the formation of liposomes in solution. The composition is useful for treatment of subcutaneous fat deposits, liver diseases, striae albicantes, striae atrophicae, cellulite, and decreased skin turgor, and the different etiologies therewith.

The main active ingredients are phospholipids, with phosphatidalcholine being the most important. Phosphatidalcholine and its pharmaceutically acceptable derivatives and related compounds have the biological properties of stimulating lipase production in adipose tissue. Lipase and it's related compounds stimulate the lysis, destruction, and reduction of the amount of adipose cells. Lipase also stimulates the lipolysis of fatty material contained within adipose tissue.

In the 1990's the World health Organization (WHO) identified obesity as an epidemic of mass proportions. The United States Center for Disease Control (CDC) estimates that about 300,000 Americans die annually from obesity-related illnesses. The United States Surgeon General has determined, in 2004, that there is now an epidemic of adult onset diabetes in children with an onset of about age 10. It is feared that we may be approaching a time where the present young generation may start passing away before their parents in significant numbers. Obesity is a serious and intractable health hazard. The vitality of our nation is a stake.

The underlying problem is that the body has an internal set point of how much fat composition is optimal. This set point is basically determined by the numbers of adipose cells present. This was determined by both genetic and environmental factors. The environmental factors include the behavior of a person's mother during pregnancy, behavior patterns during growth spurts during early childhood, and at puberty, and continuing behavioral patterns into adulthood.

The problem is further compounded in that obesity causes obesity. Changes in the production and or clearance of certain hormones is associated with increasing body mass and regional fat distribution. These hormonal changes promote further weight gain and affect the distribution of fat in humans. Included are high blood levels of insulin and cortisol. It also includes low blood levels of growth hormone. Testosterone blood levels are also altered, with them being elevated in women and depressed in men. These metabolic abnormalities promote excess fat deposits and a tendency to cause these deposits in body areas where it is harder to loose such fat deposits. These patterns are well known and are different in men and women.

There is even a very viscous hormonal feedback cycle involving cortisol. In the field of Psychoneuroendocrinology, it has been known that high cortisol levels most likely is involved in the genesis and character of primary mood disorders. There is also known to be a relationship between chronic stress and depressive disorders. Additional evidence suggests that prolonged high levels of cortisol can result in structural neuropathology resulting in more lasting behavioral change. See Kaplan and Sadock et al., Comprehensive Textbook of Psychiatry ed. V, volume 1 pages 105-106 (1989). And this cycle viscously accelerates as obesity releases more cortisol and this worsens underlying psychiatric pathology resulting in obesity. A very dangerous, unhealthy, and insidious pathological trap with significant crossover into non psychiatric pathological states and diseases.

When a person looses weight through dieting and exercise, the body changes metabolic patterns in order to retain fat. This results in a continuous cycle of dieting and weight gain. This repetitive cycle also significantly contributes to morbidity and mortality as a result of high circulating fat levels in the blood.

This biphasic injection formulation circumvents this vicious cycle. Administration of Lipolyte has the biological property of reducing the number of adipose cells in the treated areas. In combination with exercise and appropriate diet modifications, the ongoing starvation verses feasting cycle is circumvented.

The dispersion of the biphasic injection formulation of the present invention is useful in increasing the efficacy of subcutaneous injection treatment of subcutaneous fat deposits. This allows a more evenly and controlled dispersion of the formulation of the components within the adipose tissue. Tendencies of clumpy and uneven distribution of monophasic formulations is eliminated, along with the corresponding uneven reduction of subcutaneous fat. Additionally, this formulation can be used in the treatment of uneven fat deposits know as cellulite. Additionally, the even distribution of this formulation permits lower amounts of the biologically active substances to be injected for a given response.

The characteristic of this formulation allowing lower amounts of active material present in a given treatment permits in a more controlled reduction of fat deposits. This is of significance in that the reduction of these fat deposits is accomplished by the lysis and destruction of the adipose tissues present. Such destruction of even unwanted cells has further pathologic sequella and the more gradually and evenly such destruction can be accomplished diminishes the associated morbidity. In addition, with slow, gradual reduction of fat deposits, the physiologic and hormone mediated response of the bodies feast verses famine cycle can be circumvented.

The lysis of subcutaneous adipose cells constitutes an injury physiologically. A stress response results which can result in profound metabolic abnormalities. It is for this reason that these physiological injuries of lysis proceed in a highly controlled manner as facilitated by the unique composition and properties of this biphasic injection formulation. As a result a more limited stress load can be imposed at during a given time period, thus circumventing a significant stress response. Additionally, components of this biphasic injection formulation help reduce and treat what stress response results.

A stress response results in profound metabolic abnormalities following the release of inflammatory mediators and the development of an abnormal “stress induced” hormonal environment. An increase in the proinflammatory cytokines TNF, II.sub6, IL.sub.8 and increased oxidant activity, result in further increased cell damage and protein degradation. The cell damage and protein degradation result in an increase in endogenous catabolic hormones. Catalysis of adipose tissue is an objective of treatment with this formulation. However, it is desirable that such catabolic action be localized, controlled, and self limiting.

An increase in the endogenous hormones, such as catechols, cortisol, and lipase, and a decrease in normal endogenous anabolic activity can lead to a large net protein loss, if this occurs in a large quantity. In addition, a profound increase in cell energy demands arises, markedly increasing the need for nutrient utilization, while at the same time energy production becomes very inefficient. This degree of increase in metabolic rate varies with the degree of systemic injury. To this end it is imperative that the quantity of systemic injury be limited and highly controlled. The properties of this biphasic injection formulation greatly facilitate these ends by more even dispersion of the involved active substances. This limits their total quantity in a given area permitting such catabolic reactions to be more controlled, localized, and self limiting. The decrease in total adipose tissue can be broken into much more controlled limiting steps. As a result, this treatment can proceed with little or no systemic side effects.

Peak hypermetabolism and increased energy demand begins immediately post injury. It is therefore appropriate to give nutrition support to the treated area involving large amounts of tissue. An entire spectrum of abnormalities can be seen post injury including infection and also inflammation as a manifestation of the host “stress response.” If uncontrolled, this process becomes auto destructive. Support of the metabolic machinery is necessary to prevent further spread of this process.

Lean body mass (LBM) makes up 70% of body weight, with 75% of LBM being water and 20% of the LBM being protein. Almost all protein content of the body is in the LBM compartment. Each protein molecule has a functional role in maintaining homeostasis.

The degree of lean body mass, or body protein loss, in a catabolic state is correlative to morbidity and mortality. LBM loss exceeding 10% of total, can occur within a week after sever injury, despite provision of appropriate macro nutrients, carbohydrates, fat, and protein. A loss of lean mass exceeding 10% of total body protein will result in an immune deficiency state. When losses exceed 15% of body protein, there is also a marked increase in infections, sever weakness, skin breakdown (pressure sores), and the absence of wound healing. A loss of LBM exceeding 40% is usually fatal.

Excess oxidant release is known to produce further tissue injury. Oxidants are very unstable metabolites of oxygen released by inflammatory cells when activated. The oxidants injure tissue by reacting with the cell membrane lipid layers and tissue proteins, thereby producing biochemical damage via the oxidation process. Oxidation of lipids, particularly those of the cell membrane, result in a self-perpetuating process known as lipid peroxidation. Lipid peroxidation results in an alteration in cell membrane function. Post injury red cell hemolysis is caused by oxidant cell membrane injury. However, it is the goal of this biphasic injection formulation to cause peroxidation of the adipose cell membranes, but to limit such damage to them. Further lipolysis of the released contents of the lysed adipose cells in vitro is also a goal of this biphasic injection formulation. However, it is also a goal to limit such peroxidation and lipolysis to adipose cells and their contents only. The properties of the biphasic composition of this invention limit these processes to small localities surrounding the injection sites. This is the result of the controlled dispersion properties of this formulation. In addition, the formulation contains sodium chloride, a mild sclerosing agent. This sclerosis of localized tissue has the biological effect of compartmentalizing these chemical processes to small confined local areas, thus limiting the spread of these chemical reactions. This prevents the production of a self perpetuating reaction which will then spread to other areas and tissues.

Additionally, the biphasic formulation can also become a carrier of micro nutrients into the localized area of lipolysis. The support of such micro nutrients can further limit the spread of such cascading catabolic processes.

Proteins attacked by oxidants will be denatured, thus rendering them inactive with respect to their normal biological functions. This becomes of particular concern with respect to enzymes and interstitial proteins. However, the sclerosing action of the formulation results in some of the interstitial proteins adhering in what may be described as similar to scar tissue formation. As a result, the catabolic reactions tend to become compartmentalized as described above. Many other processes are also affected by oxidant damage. Antioxidant administration has been shown to attenuate these processes.

The inventors have devised a therapeutic biphasic lecithin based injection formulation useful for treating patients with subcutaneous fat deposits, liver diseases, striae albicantes, striae atrophicae, cellulite, and decreased skin turgor, and the different etiologies therewith. Each of the components serves to provide nutrients and other biological functions within the physiological system of the patient.

Lecithin is the most abundant phospholipid in the body. It is a fatty food substance. It is a structural material in every cell of the body. It forms 30% of the dry weight of the brain. It is an important constituent of endocrine glands, muscles, the heart, kidneys, liver, and blood. It occurs naturally in many foods including vegetable oils, eggs, whole grain cereals, soybeans, liver, and milk. It is also synthesized in the body, primarily in the liver.

It has properties which allow it to emulsify oils and cholesterol, making them soluble and transportable in aqueous media. These proprieties allow it to break up cholesterol and other lipid compounds into smaller particles more easily transported, assimilated and metabolized. As such, it is included in the bile produced by the liver which makes fats soluble in the small intestine, and after these fats are absorbed through the intestinal wall, the lecithin is included in the enterohepatic recirculation recycling process. It is an extremely important factor in the digestion and oxidation of fats. The disease process atherosclerosis is characterized by increased cholesterol and decreased lecithin in the blood.

Lecithin has been used in the treatment of atherosclerosis, xanthelasma, anxiety, depression, immunodeficiency, acne, eczema, psoriasis, diabetes, exhaustion, and impotence. It is a primary source of phosphatidylcholine. Phosphatidylcholine from less than 10% to over 96% of lecithin.

Phosphatidylcholine is a primary dietary source of choline, is composed of a phosphate group, 2 fatty acids, and choline. It is the composition of the fatty acids that determines it's value in promoting health. After ingestion, most is broken down into choline, glycerol free fatty acids, and the phosphate group. Some is incorporated intact into cell membranes. However, most cell membrane phospholipids are synthesized from these and other components for use in cell membranes. Although choline can be manufactured in humans form methionine or serine, it has recently been designated an essential nutrient.

Choline is required for the proper metabolism of fats and facilitates the movement of fats in and out of cells. In the human body, it is a methyl donor. This is an extremely important metabolic step in the functions of the liver and other metabolic machinery of the human body. It is vital in liver function due to it's role in the lipotropic effect which involves the export of fat from the liver. Without adequate choline, fats become trapped in the liver and as a result block many metabolic steps. Stagnation of these key metabolic pathways leads to serious liver disorders including cirrhosis. The functioning of similar metabolic pathways is vital for the transport of fats into and out of adipose tissue, and important consideration in the operation of this biphasic injection formulation.

Choline is needed for cell membrane integrity. It plays a critical role in the manufacture of primary cell membrane components including phosphatidylcholine and sphingomyelin. It is a main structural support of cell membranes. Cell membranes are dynamic molecular sheets on which most biochemical life processes occur. Phosphatidylcholine comprises about 40% of the total membrane phospholipids. It is important for homeostatic regulation of membrane fluidity. It is an important mediator of prostaglandin and eicosanoid cellular messenger functions and for support of signal transduction from the cell's exterior to its interior.

The operation of cell membranes is the key to all life processes as we understand them. These functions are crucial to the functioning of this biphasic injection formulation. The chemical signaling from the exterior to the interior of the cell is vital in the triggering of the release of lipase, and it's related materials, upon which a basic function of this biphasic injection formulation depends. But these functions extend far beyond the initial scope of this biphasic injection formulation for the treatment of subcutaneous fat deposits.

The proper functioning of cell membranes is vital for the cell to “speak or communicate” with other cells in it's proximate environment. One of the primary biological failures behind the cluster of diseases known as cancer is a failure of cells to normally communicate within their proximate environment. This communication is necessary to regulate the rate of cell division. Such a failure of communication from cell to cell results in unregulated cell division where each cell has become “an island unto itself.” Though there are many pathological conditions underlying cancer, all cancers share this fundamental communication failure. As a result, choline is vital in the normal function of cells and probably plays significant roles in the pathology of cancer. There is a strong possibility that proper maintenance of normal choline composition of cell membranes will play a role in the prevention and treatment of many cancers.

Choline is essential in the synthesis of acetylcholine which is essential in many brain, neuronal, and other chemical processes of life.

Phosphatidylcholine is the main lipid constituent of the lipoprotein particles circulating in the blood. It increases the solubility of cholesterol thus lowering cholesterol levels, removing cholesterol from tissue deposits, and inhibits platelet aggregation. All of these processes contribute to atherosclerosis.

Phosphatidylcholine's amphipathic properties make it a necessary micellizing constituent of bile. It has surfactant properties making it a protector of the epithelial-luminal interfaces of both the lungs and GI tract. It is a precursor for other phospholipids and their components as described above. It additionally has antioxidant properties.

The health of the cell membrane is synonymous with health of the entire organism. Toxins have an affinity for fatty acids; they literally take up residence in the lipid environment and in so doing, weaken and disrupt metabolic processes. The probably result is early apoptosis, premature death of the cell. Generally, normal mitosis provides for new cellular growth to maintain the health of the body. However, toxicity's affinity for lipids can easily redistribute toxins and diseased toxic lipids into new growth. In a healthy state with adequate glutathione and ascorbate to bind toxins before they take up new residence, the body can keep up with the bad guys under control. However, if defenses are weak, toxins can continually be redistributed and eventually hide in the CNS and bone where regeneration is slow.

Detoxification of neurotoxins requires that the cell membrane is nourished with balanced essential fatty acids and supportive phospholipids. Phosphatidylcholine is the main lipid constituent of cell membranes and assists the 33,000 square meters of liver cell membrane to be protected from toxicity and infection. The liver should play a pivotal role in detoxification but due to its fatty acid content and the lipid soluble characteristics of neurotoxins, lipid based interventions, such as possible with this biphasic injection formulation, are required to impact toxic burdens. Once the liver has been damaged it can no longer metabolize fats normally. Pools of lipids are then deposited within the hepatocytes throughout the liver. Beta oxidation of fatty acids is suppressed impairing detoxification and prostaglandin production. However, research has shown that phosphatidylcholine protects the liver against damage from alcohol, pharmaceuticals, environmental pollutants, xenobiotics, and infection due to viral, bacterial, and fungal manifestations.

The widespread biological properties of lecithin and phosphatidalcholine indicate the importance of these micro nutrient compositions of this biphasic injection formulation to the basic life processes and maintenance of homeostasis. Additionally, these properties are also vital to protect the body from possible systemic complications from the injury of lysis of adipose tissue. It additionally mitigates against the further spread of this damage from the localized treatment area. It also facilitates the restoration of the remaining cells to a normally healthy state. Additionally, toxins developed are relatively contained within the small treated area.

Hydrogenated lecithin and phosphatidalcholine are simply more stable variants of their related compounds.

Lysolecithin is a lecithin molecule form which the alpha fatty acid has been removed. It has strong hemolytic properties and exists in trace amounts in the pancreas. A lysophosphatide, as in lysophosphatidylcholine, also has one fatty acid molecule removed. It would as a result also be hemolytic. Both of these compounds are most likely involved in the lipolysis of the adipose cells.

Lysophosphatides are produced by the action of injected cobra venom on phospholipids. Their resulting hemolytic properties are part of the pathological response to cobra envenomation. This hemolysis action is the result of disruption of the cell membrane of the red blood cells. This action closely approximates the actions involved in lipolysis. It is an action of the properties of the Lysophosphatides to include lipolysis of adipose cells. In addition to the stimulation and release of lipase products, these Lysophosphatides directly contribute to this action.

It is important that the reader understand that these actions are diametrically opposed to the protective, homeostatic, antioxidant, anti toxic effect of the phospholipids. This biphasic injection formulation functions by the action of both of these antagonistic qualities. Additionally, the sclerosing actions of other components “wall off” these sites of reaction to make them self limiting and confined to a controlled area. The management of these antagonistic actions of such a preparation as embodied by this biphasic injection formulation takes the skill and clinical experience of a physician with experience in such art.

Tocopherol is one of the forms of vitamin E. It is an antioxidant and protects lipids from free radical oxidation, thus preventing the formation of toxic metabolites. It is used in many skin care products and has numerous effects in the promotion of skin health.

Alpha lipoic acid is one of the most powerful antioxidant and antiinflammatory agents known for use in humans. It potentates the actions of vitamin C and vitamin E and in addition protects these vitamins from damage. It is both fat and water soluble. It strengthens the immune system and protects the mitochondria and cellular DNA. With it's mitochondrial effects, it actually increases the metabolism of cells with low metabolism. It is the only known biological agent that can do this. It's clinical effects are important including promotion and acceleration of healing, reduction of inflammation, reduction of scaring. It increases skin tone, health, and decreases wrinkles.

Ascorbyl palmitate is a vitamin C ester which makes it lipid soluble. It is also an antioxidant and free radical scavenger. It boosts the immune system, promotes energy production and is essential to the operation of the nervous system. It repairs sun damaged skin, and damaged collagen. As a result it decreases wrinkles and decreases skin sagging due to depleted or damaged collagen. It reduces inflammation.

Water is the most common compound of our bodies and is the foundation to life as we understand it.

Sodium chloride is a salt that is ubiquitous to life. Sodium is one of the most commonly transferred ion through the gates of various membranes of cells. It is the most common ion used in all of the “switches” of the body. Most neurotransmitters upon binding to their respective synaptic sites involve the shifting of sodium across a membrane. It is also a mild sclerosing agent and is used as such in this biphasic injection formulation. It is also a component of the buffering action of this biphasic injection formulation.

The components of the buffering action of this biphasic injection formulation include sodium chloride, water, phosphatidylcholine, hydrogenated phosphatidylcholine, tocopherol, lecithin, lysophosphatidylcholine, hydrogenated lecithin. These components also comprise a stabilizer. Tocopherol, alpha lipoic acid, and ascorbyl palmitate are antioxidants.

As stated above, the subcutaneous fat removal injection products currently marketed are primarily monophasic lecithin based mixtures of lipids. The biphasic nature of this biphasic injection formulation allows better and more even distribution of biologically active substances. The sclerosing action limits the actions of the biphasic injection formulation to highly predetermined and controlled locations. The actions are thus localized and self limiting. Components of this biphasic injection formulation promote the healing of remaining tissue and reduce the production and dissemination of toxic metabolites. Further, the above mentioned sclerosing action promotes increased skin tone and turgor, therefore decreasing skin sagging and wrinkling resulting from the use of currently marketed preparations. Additionally, this biphasic injection formulation can be formulated as a delivery vehicle for other biologically active substances. In addition, the components of the lipid and aqueous phases, in and of themselves have therapeutic properties valuable in the treatment of many human disease conditions as elaborated above.

While I believe the operations of this invention occur as described above, I don't wish to be limited and or bound by these explanations.

Preferred Embodiments

The first preferred composition are those of the following general formula:
Aqueous phase approximately comprises about 50% of the total;
Sodium chloride approximately comprises about 0.25% of the aqueous phase;
Lipid phase approximately comprises about 50% of the total;
Phosphatidylcholine approximately comprises about 90-96% of the lipid phase;
Lysophosphatidylcholine approximately comprises about 0.0-6% of the lipid phase;
Lecithin approximately comprises about 0.0-6% of the lipid phase;
Lysolecithin approximately comprises about 0.0-6% of the lipid phase;
Tocopherol approximately comprises about 0.0-1% of the lipid phase;
Hydrogenated variants of the above compounds comprise 0.0-100% of the given compound.

This first preferred composition is suitable for subcutaneous injection only. It contains hemolytic and lipolytic components that must not be delivered intravenously or intramuscularly except under rare conditions as deemed necessary by a physician. This composition of the formulations of this biphasic injection formulation is to be used primarily for treatment of subcutaneous fat deposits, cellulite deposits, decreased skin turgor, striae atrophicae, and striae albicantes.

This basic formulation may be altered by the addition of predetermined amounts of alpha lipoic acid, ascorbyl palmitate, and other micro nutrients as determined necessary by the physician treating a given patient.

The second preferred composition are those of the following general formula:
Aqueous phase approximately comprises about 50% of the total;
Sodium chloride approximately comprises about 0.25% of the aqueous phase;
Lipid phase approximately comprises about 50% of the total;
Phosphatidylcholine approximately comprises about 90-96% of the lipid phase;
Lecithin approximately comprises about 0.0-6% of the lipid phase;
Tocopherol approximately comprises about 0.0-1% of the lipid phase;
Hydrogenated variants of the above compounds comprise 0.0-100% of the given compound.

Significantly absent are the Lysophosphatidylcholine, Lysolecithin, and other Lysophosphatide variants included in the first formulation. This formulation is suitable for subcutaneous, intravenous, and intramuscular injection. It can be used for the treatment of fat deposits, cellulite deposits, decreased skin turgor, striae atrophicae, and striae albicantes. However, it can be used also as a carrier of other biologically active substances. Additionally, it can be used to treat many human ailments which are treatable by components of the this biphasic preparation as described in the above detailed descriptions.

It is important that the reader understand that the phosphatide compounds taken orally in foods and supplements most likely contain some lysophosphatide breakdown contaminants. However, during the transmural transport of these phosphatides across the small intestine lumen, they are broken down into choline, glycerol free fatty acids, and the phosphate group. These components are then reassembled into the phosphatide compounds needed without forming lysophosphatides in the process.

The subcutaneous injection of lysophosphatide variants circumvents this protection mechanism of the body, allowing the deposit of lipolytic compounds for therapeutic purposes. But the introduction of these normally dangerous compounds is done in a highly controlled and self limiting manner as described above.

This second basic formulation may be altered by the addition of predetermined amounts of alpha lipoic acid, ascorbyl palmitate, and other micro nutrients as determined necessary by the physician treating a given patient.

Many variations of these two basic preferred embodiments are possible. These other embodiments of the biphasic injection formulation will be apparent to those skilled in the art from consideration of the specification and practice of the invention disclosed herein. It is intended that the specification be considered exemplary only, with the true scope and spirit of the invention being indicated by the attached claims.

Conclusion, Ramifications, and Scope

Accordingly, the reader will see that the biphasic injection dosage formulation of this invention can be used to treat subcutaneous fat deposits, can be used to increase skin turgor, and can be used to treat subcutaneous cellulite. In addition, with the addition of other biologically active substances, the biphasic injection dosage formulation of this invention can be used as a carrier of such substances to the target tissues of a human. Furthermore, this invention comprises a coordinated program of treatment which comprised the injections, wearing a compression garment, diet modification, and an exercise program. These features increase the efficacy of treatment over just injections alone. Furthermore, the biphasic injection dosage formulation has the additional advantages in that:
it permits the thinning of the phospholipid mixture prior to injection;
it provides for increased and more uniform dispersion of the phospholipid in the target tissue;
as a result of more uniform dispersion of the injected dosage formulation, there is a marked reduction in the uneven dissolution of subcutaneous fat deposits as compared with previous products. As a result, the tendency to leave uneven, lumpy deposits under the skin is eliminated;
it provides a means of treatment of striae albicantes in a human in need of such treatment;
it provides a means of treatment of striae atrophicae in a human in need of such treatment;
it provides a means of treatment of atherosclerotic plaque accumulation in a human in need of such treatment;
it provides a means of treatment of liver disease in a human in need of such treatment;
it permits the predetermining of the pH and capacity of it's buffering action;
it provides a means of treating human ailments responsive to the biological properties of it's lipophylic components;
it provides a means of treating human ailments responsive to the biological properties of it's aqueous components.
   Although the description above contains many specificities, these should not be construed as limiting the scope of the invention but as merely providing illustrations of some of the presently preferred embodiments of this biphasic injection formulation. For example, the relative amounts of the lipid and aqueous compounds may change; biologically active materials may be carried by the formulation, etc. Other embodiments of the biphasic injection formulation will be apparent to those skilled in the art from consideration of the specification and practice of the invention disclosed herein. It is intended that the specification be considered as exemplary only, with the true scope and spirit of the invention being indicated by the appended claims and their legal equivalents, rather than by the examples given.

1. A method for reduction of adipose tissue in humans comprising injecting such humans with a dosage formulation of lecithin or its pharmaceutically acceptable derivatives in an amount effective to reduce said adipose tissue.

2. A biphasic injection dosage formulation comprising:
(a) an aqueous phase comprising an aqueous solution of water and sodium chloride or its pharmaceutically acceptable derivatives;
(b) a lipidic phase made by preparing a solution of the following or their pharmaceutically acceptable derivatives;
(i) phosphatidylcholine;
(ii) hydrogenated phosphatidylcholine;
(iii) lysophosphatidylcholine;
(iv) tocopherol;
(v) lecithin;
(vi) hydrogenated lecithin;
(c) said phosphatidylcholine or its pharmaceutically acceptable derivatives being present in an effective amount and having the biological properties of causing adipose cells and related tissue to release lipase and related substances, said lipase and related substances having the biological properties of the lysis, destruction and reduction of the amount of adipose cells in a given region, said lipase and related substances having the biological properties resulting in lipolysis of fatty material contained within said adipose cells;
(d) said hydrogenated phosphatidylcholine or its pharmaceutically acceptable derivatives being present in an effective amount and having the biological properties of causing adipose cells and related tissue to release lipase and related substances, said lipase and related substances having the biological properties of the lysis, destruction, reduction of the amount of adipose cells in a given region, said lipase and related substances having the biological properties resulting in lipolysis of fatty material contained within said adipose cells;
(e) said lysophosphatidylcholine or its pharmaceutically acceptable derivatives being present in an effective amount and having the biological properties of causing adipose cells and related tissue to release lipase and related substances, said lipase and related substances having the biological properties of the lysis, destruction and reduction of the amount of adipose cells in a given region, said lipase and related substances having the biological properties resulting in lipolysis of fatty material contained within said adipose cells;
(f) said tocopherol or its pharmaceutically acceptable derivatives being present in an effective amount and having the biological properties of causing adipose cells and related tissue to release lipase and related substances, said lipase and related substances having the biological properties of the lysis, destruction, and reduction of the amount of adipose cells in a given region, said lipase and related substances having the biological properties resulting in lipolysis of fatty material contained within said adipose cells;
(g) said lecithin or its pharmaceutically acceptable derivatives being present in an effective amount and having the biological properties of causing adipose cells and related tissue to release lipase and related substances, said lipase and related substances having the biological properties of the lysis, destruction, and reduction of the amount of adipose cells in a given region, said lipase and related substances having the biological properties resulting in lipolysis of fatty material contained within said adipose cells;
(h) said hydrogenated lecithin or its pharmaceutically acceptable derivatives being present in an effective amount and having the biological properties of causing adipose cells and related tissue to release lipase and related substances, said lipase and related substances having the biological properties of the lysis, destruction, and reduction of the amount of adipose cells in a given region, said lipase and related substances having the biological properties resulting in lipolysis of fatty material contained within said adipose cells;
(i) thereby resulting in the reduction of the amount of adipose tissue present in a treated area;
(j) thereby resulting in the reduction of the number of adipose cells present in the treated area.

3. The means of claim 2, wherein said injection is subcutaneous injection.

4. The means of claim 2, wherein said injection is intravenous injection.

5. The means of claim 2, wherein said injection is intramuscular injection.

6. The aqueous based composition of claim 2, containing a predetermined amount of sodium chloride from 0.0 to 100 percent.

7. The aqueous based composition of claim 2, containing a predetermined amount of water from 0.0 to 100 percent.

8. The lipid based composition of claim 2, containing a predetermined amount of phosphatidylcholine from 0.0 to 100 percent.

9. The lipid based composition of claim 2, containing a predetermined amount of hydrogenated phosphatidylcholine from 0.0 to 100 percent.

10. The lipid based composition of claim 2, containing a predetermined amount of lysophosphatidylcholine from 0.0 to 100 percent.

11. The lipid based composition of claim 2, containing a predetermined amount of tocopherol from 0.0 to 100 percent.

12. The lipid based composition of claim 2, containing a predetermined amount of lecithin from 0.0 to 100 percent.

13. The lipid based composition of claim 2, containing a predetermined amount of hydrogenated lecithin from 0.0 to 100 percent.

14. Said biphasic injection dosage formulation of claim 2, containing a predetermined amount of the aqueous phase from 0.0 to 100 percent.

15. Said biphasic injection dosage formulation of claim 2, containing a predetermined amount of said lipidic phase from 0.0 to 100 percent.

16. Said biphasic injection dosage formulation of claim 2, characterized in that it further comprises a sclerosing agent.

17. Said biphasic injection dosage formulation of claim 2, wherein said sclerosing agent is aqueous sodium chloride.

18. Said biphasic injection dosage formulation of claim 2, characterized in that it further comprises a stabilizer.

19. Said biphasic injection dosage formulation of claim 2, wherein said stabilizer is the components of said lipid phase comprising;
(a) phosphatidylcholine;
(b) hydrogenated phosphatidylcholine;
(c) lysophosphatidylcholine;
(d) tocopherol;
(e) lecithin;
(f) hydrogenated lecithin.

20. Said biphasic injection dosage formulation of claim 2, characterized in that it further comprises a buffer.

21. Said biphasic injection dosage formulation of claim 2, wherein said buffer is comprised of:
(a) sodium chloride;
(b) phosphatidylcholine;
(c) hydrogenated phosphatidylcholine;
(d) Iysophosphatidylcholine;
(e) tocopherol;
(f) lecithin;
(g) hydrogenated lecithin;
(h) water.

22. Said biphasic injection dosage formulation of claim 2, wherein the hydrogen ion concentration of said buffer is set by predetermining the relative concentrations of the components of said buffer.

23. Said biphasic injection dosage formulation of claim 2, wherein the capacity of said buffer is set by predetermining the relative concentrations of the components of said buffer.

24. Said biphasic injection dosage formulation of claim 2, characterized in that it further comprises an antioxidant.

25. Said biphasic injection dosage formulation of claim 2, wherein said antioxidant is tocopherol.

26. A means of treating subcutaneous adipose tissue accumulation in a human in need of such treatment comprising administration of a predetermined amount of said biphasic dosage formulation of claim 2 by said injection.

27. A means of administering biologically active substances into tissue by said injection using said biphasic dosage formulation of claim 2 as a carrier of said biologically active substances.

28. A means of treating decreased skin turgor in a human in need of such treatment comprising administration of a predetermined amount of said biphasic dosage formulation of claim 2 by said injection.

29. A means of treating striae atrophicae in a human in need of such treatment comprising administration of a predetermined amount of said biphasic dosage formulation of claim 2 by said injection.

30. A means of treating striae albicantes in a human in need of such treatment comprising administration of a predetermined amount of said biphasic dosage formulation of claim 2 by said injection.

31. A means of treating subcutaneous adipose tissue accumulation in a human in need of such treatment comprising administering a pharmaceutically effective amount of the biphasic dosage formulation of claim 2.

32. A means of treating subcutaneous cellulite accumulation in a human in need of such treatment comprising administering a pharmaceutically effective amount of the biphasic dosage formulation of claim 2.

33. A means of treating atherosclerotic plaque accumulation in a human in need of such treatment comprising administering a pharmaceutically effective amount of the biphasic dosage formulation of claim 2.

34. A means of treating liver disease in a human in need of such treatment comprising administering a pharmaceutically effective amount of the biphasic dosage formulation of claim 2.

35. Said biphasic injection dosage formulation of claim 2, containing a predetermined amount of anesthetic from 0.0 to 100 percent.

36. Said anesthetic of claim 35, wherein the anesthetic is lidocaine.

37. Said anesthetic of claim 35, wherein the anesthetic is bipivacaine.

38. A means of treating subcutaneous adipose tissue accumulation in a human in need of such treatment comprising:
(a) said injection of said biphasic injection dosage formulation of claim 2;
(b) application of a compression garment;
(c) a predetermined exercise program;
(d) a predetermined diet regimen.

39. The means of claim 38, wherein said compression garment is to be worn from 0 to 24 hours in a 24 hour time period.

40. The means of claim 38, wherein said compression garment is to be worn for a predetermined number of 24 hour time periods.

41. The means of claim 38, wherein said exercise program is to be predetermined by a practitioner skilled in such art.

42. The means of claim 38, wherein said diet regimen is to be predetermined by a practitioner skilled in such art.

43. A biphasic injection dosage formulation comprising:
(a) an aqueous phase comprising an aqueous solution of water and sodium chloride or its pharmaceutically acceptable derivatives;
(b) a lipidic phase made by preparing a solution of the following or their pharmaceutically acceptable derivatives;
(i) phosphatidylcholine;
(ii) hydrogenated phosphatidylcholine;
(iii) lysophosphatidylcholine;
(iv) tocopherol;
(v) lecithin;
(vi) hydrogenated lecithin;
(c) said biphasic dosage formulation provides a means of injection delivery of soluble biologically active substances.

44. The aqueous based composition of claim 43, containing a predetermined amount of sodium chloride from 0.0 to 100 percent.

45. The aqueous based composition of claim 43, containing a predetermined amount of water from 0.0 to 100 percent.

46. The lipid based composition of claim 43, containing a predetermined amount of phosphatidylcholine from 0.0 to 100 percent.

47. The lipid based composition of claim 43, containing a predetermined amount of hydrogenated phosphatidylcholine from 0.0 to 100 percent.

48. The lipid based composition of claim 43, containing a predetermined amount of lysophosphatidylcholine from 0.0 to 100 percent.

49. The lipid based composition of claim 43, containing a predetermined amount of tocopherol from 0.0 to 100 percent.

50. The lipid based composition of claim 43, containing a predetermined amount of lecithin from 0.0 to 100 percent.

51. The lipid based composition of claim 43, containing a predetermined amount of hydrogenated lecithin from 0.0 to 100 percent.

52. Said biphasic injection dosage formulation of claim 43, containing a predetermined amount of the aqueous phase from 0.0 to 100 percent.

53. Said biphasic injection dosage formulation of claim 43, containing a predetermined amount of the lipidic phase from 0.0 to 100 percent.

54. Said biphasic injection dosage formulation of claim 43, characterized in that it further comprises a sclerosing agent.

55. Said biphasic injection dosage formulation of claim 43, wherein said sclerosing agent is aqueous sodium chloride.

56. Said biphasic injection dosage formulation of claim 43, characterized in that it further comprises a stabilizer.

57. Said biphasic injection dosage formulation of claim 43, wherein said stabilizer is the components of the lipid phase comprising;
(a) phosphatidylcholine;
(b) hydrogenated phosphatidylcholine;
(c) lysophosphatidylcholine;
(d) tocopherol;
(e) lecithin;
(f) hydrogenated lecithin.

58. Said biphasic injection dosage formulation of claim 43, characterized in that it further comprises a buffer.

59. Said biphasic injection dosage formulation of claim 43, wherein said buffer is comprised of:
(a) sodium chloride;
(b) phosphatidylcholine;
(c) hydrogenated phosphatidylcholine;
(d) lysophosphatidylcholine;
(e) tocopherol;
(f) lecithin;
(g) hydrogenated lecithin;
(h) water.

60. Said biphasic injection dosage formulation of claim 43, wherein the hydrogen ion concentration of said buffer is set by predetermining the relative concentrations of the components of said buffer.

61. Said biphasic injection dosage formulation of claim 43, wherein the capacity of said buffer is set by predetermining the relative concentrations of the components of said buffer.

62. Said biphasic injection dosage formulation of claim 43, characterized in that it further comprises an antioxidant.

63. Said biphasic injection dosage formulation of claim 43, wherein said antioxidant is tocopherol.

64. A means of administering biologically active substances into tissue by said injection using said biphasic injection dosage formulation of claim 43 as a carrier of said biologically active substances.

65. A means of treating atherosclerotic plaque accumulation in a human in need of such treatment comprising administering a pharmaceutically effective amount of said biphasic injection dosage formulation of claim 43.

66. A means of treating liver disease in a human in need of such treatment comprising administering a pharmaceutically effective amount of said biphasic injection dosage formulation of claim 43.

67. The means of claim 43, wherein said injection is subcutaneous injection.

68. The means of claim 43, wherein said injection is intravenous injection.

69. The means of claim 43, wherein said injection is intramuscular injection.

70. Said biphasic injection dosage formulation of claim 43, containing a predetermined amount of anesthetic from 0.0 to 100 percent.

71. Said anesthetic of claim 70, wherein the anesthetic is lidocaine.

72. Said anesthetic of claim 70, wherein the anesthetic is bipivacaine.

73. A means of treating subcutaneous adipose tissue accumulation in a human in need of such treatment comprising administration of a predetermined amount of said biphasic dosage formulation of claim 43 by injection.

74. A means of administering biologically active substances by using said biphasic dosage formulation of claim 43 as a carrier of said biologically active substances.

75. A means of treating subcutaneous cellulite accumulation in a human in need of such treatment comprising administering a pharmaceutically effective amount of the biphasic dosage formulation of claim 43.
